(19) United States
(12) Patent Application Publication
(10) Pub. No.: US 2024/0158569 A1
(43) Pub. Date: May 16, 2024

(54) Catalyst Systems for Ring-Opening Polymerization Processes to Produce Syndiotactic Polymer

(71) Applicant: ExxonMobil Technology and Engineering Company, Baytown, TX (US)

(72) Inventors: Gursu Culcu, Humble, TX (US); Catherine A. Faler, Houston, TX (US); Sarah J. Mattler, League City, TX (US); Pamela J. Wright, Easton, NJ (US); Carlos R. Lopez-Barron, Houston, TX (US); Matthew W. Holtcamp, Huffman, TX (US)

(21) Appl. No.: 18/491,022

(22) Filed: Oct. 20, 2023

Related U.S. Application Data
(60) Provisional application No. 63/380,921, filed on Oct. 25, 2022.

(51) Int. Cl. C08G 63/08; C07C 43/23; C07F 7/00; C08G 63/87

(52) U.S. Cl. CPC C08G 63/08; C07C 43/23; C07F 7/003; C08G 63/87

(57) ABSTRACT

Catalyst systems useful in ring-opening polymerization of β-butyrolactone to produce syndiotactic PHB are provided. The catalyst systems include a ligand of the structural Formula I, Formula II, or Formula III and a Group 3 metal or metal precursor such as yttrium. The present catalyst systems are useful in adjusting syndiotacticity and thereby lowering the Tm of PHB for use in commercial applications.

### BACKGROUND TO RELATED APPLICATIONS

This application claims the benefit of and priority to U.S. Provisional Application No. 63/380,921 filed Oct. 25, 2022, the disclosure of which is incorporated herein by reference.

### FIELD

The present disclosure relates to ring-opening polymerization of lactones and catalyst systems for use in ring-opening polymerization that produce syndiotactic polymers.

### BACKGROUND

Various catalyst systems have been described for use in ROP of lactones. In commercial ring-opening polymerization, the catalyst system must be active at a low temperature and produce polymers having an appropriate melting temperature without a reduction in strength and other mechanical properties or impact to the crystallinity of the polymer.

### SUMMARY

Provided herein are catalyst systems comprising a ligand of the structural Formula I

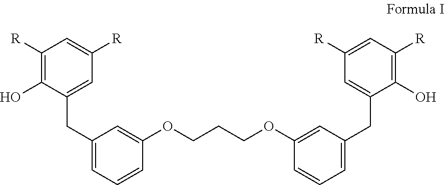

and a Group 3 metal precursor. For the ligand of the structural Formula I, R is selected from the group of hydrogen, alkyl and substituted alkyl.

Further provided are methods of ring-opening polymerization of cyclic ethers and esters comprising mixing the catalyst system with one or more lactones at room temperature to provide a syndiotactic polymer having a melting temperature (“Tm”) of between 85° C. and 110° C. and a Pr of between .55 and .70.

Also provided are methods of in situ ring-opening polymerization of β-butyrolactone comprising adding a metal precursor comprising a Group 3 metal in a solvent to a catalyst solution comprising a ligand of the structural Formula I. β-butyrolactone is added to the catalyst solution to produce syndiotactic PHB having a Pr of at least .55. The catalyst solution is mixed for at least 12 hours when PHB is separated from the catalyst solution.

These and other features and attributes of the disclosed catalyst complexes and methods of the present disclosure and their advantageous applications and/or uses will be apparent from the detailed description which follows.

### BRIEF DESCRIPTION OF DRAWINGS

To assist those of ordinary skill in the relevant art in making and using the subject matter hereof, reference is made to the appended drawings, wherein:

FIG. 1A shows the differential scanning calorimetry (“DSC”) data for the polymer of Entry 1.

FIG. 1B shows the DSC data for the polymer of Entry 2.

FIG. 1C shows the DSC data for the polymer of Entry 4.

FIG. 2A shows the ¹³C NMR spectra (carbonyl region) for the polymer of Entry 1.

FIG. 2B shows the ¹³C NMR spectra (methylene region) for the polymer of Entry 1.

FIG. 2C shows the ¹³C NMR spectra (carbonyl region) for the polymer of Entry 2.

FIG. 2D shows the ¹³C NMR spectra (methylene region) for the polymer of Entry 2.

FIG. 2E shows the ¹³C NMR spectra (carbonyl region) for the polymer of Entry 3.

FIG. 2F shows the ¹³C NMR spectra (methylene region) for the polymer of Entry 3.

FIG. 2G shows the ¹³C NMR spectra (carbonyl region) for the polymer of Entry 4.

FIG. 2H shows the ¹³C NMR spectra (methylene region) for the polymer of Entry 4.

FIG. 3A shows the ¹H NMR of the isolated yttrium complex described in Example 2.

FIG. 3B shows the COSY NMR spectrum of the isolated yttrium complex described in Example 2.

FIG. 4A shows the ¹H NMR of the PHB produced by the isolated complex of Example 3.

FIG. 4B shows the ¹³C NMR of the PHB produced by the isolated complex of Example 3.

### DETAILED DESCRIPTION

Provided herein are catalyst systems to produce poly(3-hydroxyakanoates) of varying tacticity. The present catalyst systems comprise a bisphenol ligand and an yttrium precursor, Y[N(SiHMe2)]₃(THF)₂. As described in the examples, PHB is made in situ with an yttrium precursor, Y[N(SiHMe2)]₃(THF)₂, in a solvent that is added to a catalyst solution at room temperature comprising the ligand. The catalyst solution is mixed (by stirring, blending, or swirling and the like) and β-butyrolactone added. Mixing of the catalyst solution continues for at least 3 minutes. PHB is then isolated. PHB can be characterized by NMR, GPC, and DSC methods.

The present catalyst systems are useful in ring-opening polymerization of β-lactones and are used to tune (or otherwise adjust) the melting temperature of the PHB produced while maintaining mechanical (tensile) and thermal properties as well as crystallinity.

### Definitions

The term “alkenyl” means a straight-chain, branched-chain, or cyclic hydrocarbon radical having one or more double bonds. These alkenyl radicals are optionally substituted. Examples of suitable alkenyl radicals include ethenyl, propenyl, allyl, 1,4-butadienyl cyclopropenyl, cyclobutenyl, cyclopentenyl, cyclohexenyl, cyclooctenyl, including their substituted analogues.

The term “alkyl” as used herein refers to a branched or unbranched saturated hydrocarbon group having 1 to about 50 carbon atoms, such as methyl, ethyl, n-propyl, isopropyl, n-butyl, isobutyl, sec-butyl, t-butyl, octyl, decyl, and the like, as well as cycloalkyl groups such as cyclopentyl, cyclohexyl and the like. Generally, alkyl groups contain 1 to about 20 carbon atoms.

The term “alkoxy” or “alkoxide” means an alkyl ether radical wherein the term alkyl is as defined above. Examples of suitable alkyl ether radicals include methoxy, ethoxy, n-propoxy, iso-propoxy, n-butoxy, iso-butoxy, sec-butoxy, tert-butoxy, and phenoxyl.

The term “alkynyl” as used herein refers to a branched or unbranched, cyclic, or acyclic hydrocarbon group containing 2 to about 50 carbon atoms and at least one triple bond, such as ethynyl, n-propynyl, isopropynyl, n-butynyl, isobutynyl, octynyl, decynyl, and the like. Generally, alkynyl groups herein may have 2 to about 20 carbon atoms.

The term “aromatic” includes compounds having unsaturation delocalized across several bonds around a ring. The term “aryl” as used herein refers to a group containing an aromatic ring. Aryl groups include groups containing a single aromatic ring or multiple aromatic rings that are fused together, linked covalently, or linked to a common group such as a methylene or ethylene moiety. More specific aryl groups contain one aromatic ring or two or three fused or linked aromatic rings, e.g., phenyl, naphthyl, biphenyl, anthracenyl, or phenanthrenyl. In particular embodiments, aryl substituents include 1 to about 200 atoms other than hydrogen, typically 1 to about 50 atoms other than hydrogen, and specifically 1 to about 20 atoms other than hydrogen. In some embodiments herein, multi-ring moieties are substituents and as such the multi-ring moiety can be attached at an appropriate atom. For example, “naphthyl” can be 1-naphthyl or 2-naphthyl; “anthracenyl” can be 1-anthracenyl, 2-anthracenyl or 9-anthracenyl; and “phenanthrenyl” can be 1-phenanthrenyl, 2-phenanthrenyl, 3-phenanthrenyl, 4-phenanthrenyl or 9-phenanthrenyl.

The term “aromatic ring” is a cyclic structure containing conjugated pi bonds with unhybridized p orbitals that overlap to form a continuous loop resulting in a lower energy due to delocalization of electrons.

The term “aryl” or “aryl group” means a carbon-containing aromatic ring and the substituted variants thereof can include phenyl, 2-methyl-phenyl, xylyl, 4-bromo-xylyl. Likewise, heteroaryl means an aryl group where a ring carbon atom (or two or three ring carbon atoms) has been replaced with a heteroatom, such as N, O, or S. As used herein, the term “aromatic” also refers to pseudo aromatic heterocycles which are heterocyclic substituents that have comparable properties and structures (nearly planar) to aromatic heterocyclic ligands, but are not by definition aromatic; likewise, the term aromatic also refers to substituted aromatics.

The terms “aryloxy” and “aryloxide” mean an aryl group bound to an oxygen atom, such as an aryl ether group/radical connected to an oxygen atom and can include those where the aryl group is a C₁ to C₁₀ hydrocarbyl. Examples of suitable aryloxy radicals can include phenoxy, and the like.

As used herein, a “catalyst” includes a single catalyst, or multiple catalysts. Catalysts can have isomeric forms such as conformational isomers or configurational isomers. Conformational isomers include, for example, conformers and rotamers. Configurational isomers include, for example, stereoisomers.

As used herein, the terms “catalyst” and “catalyst complex” are used interchangeably.

The term “complex” is sometimes referred to as a catalyst precursor, a precatalyst, a catalyst, a catalyst compound, a transition metal compound, or a transition metal complex. These terms are used interchangeably. Activator and cocatalyst are also used interchangeably.

As used herein, a “catalyst system” includes at least one catalyst compound and an activator. A catalyst system of the present disclosure can further include a support material and an optional co-activator. For the purposes of this disclosure, when a catalyst is described as including neutral stable forms of the components, the ionic form of the component is the form that reacts with the monomers to produce polymers. Furthermore, catalysts of the present disclosure can be represented by a Formula are intended to embrace ionic forms thereof of the compounds in addition to the neutral stable forms of the compounds. Furthermore, activators of the present disclosure are intended to embrace ionic/reaction product forms thereof of the activator in addition to ionic or neutral form.

The term “continuous” means a system that operates without interruption or cessation. For example, a continuous process to produce a polymer would be one where the reactants are continually introduced into one or more reactors and polymer product is continually withdrawn during a polymerization process.

The terms “cyclo” and “cyclic” are used herein to refer to saturated or unsaturated radicals containing a single ring or multiple condensed rings. Suitable cyclic moieties include, for example, cyclopentyl, cyclohexyl, cyclooctenyl, bicyclooctyl, phenyl, naphthyl, pyrrolyl, furyl, thiophenyl, imidazolyl, and the like. Cyclic moieties can include between 3 and 200 atoms other than hydrogen, between 3 and 50 atoms other than hydrogen or between 3 and 20 atoms other than hydrogen.

As used herein, and unless otherwise specified, the term “Cₙ” means hydrocarbon(s) having n carbon atom(s) per molecule, where n is a positive integer. Likewise, a “Cₘ-C_{y}” group or compound refers to a group or compound comprising carbon atoms at a total number thereof in the range from m to y. Thus, a C₁-C₄ alkyl group refers to an alkyl group that includes carbon atoms at a total number thereof in the range of 1 to 4, e.g., 1, 2, 3, and 4.

The term “divalent” as in “divalent hydrocarbyl,” “divalent alkyl,” “divalent aryl,” and the like, means and includes a hydrocarbyl, alkyl, aryl or other moiety is bonded at two points to atoms, molecules or moieties with the two bonding points being covalent bonds.

The acronym “DRI” means differential diffraction index.

The terms “group,” “radical,” and “substituent” are used interchangeably herein.

The terms “halo” and “halogen” are used in the conventional sense to refer to a chloro, bromo, fluoro or iodo radical.

More generally, the modifiers “hetero” and “heteroatom-containing”, as in “heteroalkyl” or “heteroatom-containing hydrocarbyl group” refer to a molecule or molecular fragment in which one or more carbon atoms is replaced with a heteroatom. Thus, for example, the term “heteroalkyl” refers to an alkyl substituent that is heteroatom-containing. When the term “heteroatom-containing” introduces a list of possible heteroatom-containing groups, it is intended that the term apply to every member of that group. That is, the phrase “heteroatom-containing alkyl, alkenyl and alkynyl” is to be interpreted as “heteroatom-containing alkyl, heteroatom-containing alkenyl and heteroatom-containing alkynyl.”

The term “heteroaryl” refers to an aryl radical that includes one or more heteroatoms in the aromatic ring. Specific heteroaryl groups include groups containing heteroaromatic rings such as thiophene, pyrazine, isoxazole, pyrazole, pyrrole, furan, thiazole, oxazole, imidazole, isothiazole, oxadiazole, triazole, and benzo-fused analogues of these rings, such as indole, carbazole, benzofuran, benzothiophene, benzimidiazole, benzthiazole, benzoxazoles, indazole and the like and isomers thereof, e.g., reverse isomers.

The terms “heterocycle” and “heterocyclic” refer to a cyclic radical, including ring-fused systems, including heteroaryl groups as defined below, in which one or more carbon atoms in a ring is replaced with a heteroatom—that is, an atom other than carbon, such as nitrogen, oxygen, sulfur, phosphorus, boron or silicon. Heterocycles and heterocyclic groups include saturated and unsaturated moieties, including heteroaryl groups as defined below. Specific examples of heterocycles include pyrrolidine, pyrroline, furan, tetrahydrofuran, thiophene, imidazole, oxazole, thiazole, indole, and the like, including any isomers of these. Additional heterocycles are described in Katritzky, A. R., Handbook of Heterocyclic Chemistry Pergammon Press, 1985, and Katritzky, A. R., et al., Comprehensive Heterocyclic Chemistry, Elsevier, 2d. ed., 1996.

A heterocyclic ring is a ring having a heteroatom in the ring structure as opposed to a heteroatom substituted ring where a hydrogen on a ring atom is replaced with a heteroatom. For example, tetrahydrofuran is a heterocyclic ring and 4-N,N-dimethylamino-phenyl is a heteroatom substituted ring.

A substituted heterocyclic ring is a heterocyclic ring where a hydrogen of one of the ring atoms is substituted, e.g., replaced with a hydrocarbyl, or a heteroatom containing group (as further described in the definition of “substituted” herein).

The term “hydrocarbyl” refers to hydrocarbyl radicals containing 1 to about 50 carbon atoms, specifically 1 to about 24 carbon atoms, most specifically 1 to about 16 carbon atoms, including branched or unbranched, cyclic or acyclic, saturated or unsaturated species, such as alkyl groups, alkenyl groups, aryl groups, and the like.

The terms “hydrocarbyl radical,” “hydrocarbyl group,” or “hydrocarbyl” are used interchangeably and are defined to mean a group comprising hydrogen and carbon atoms. Hydrocarbyls are CI -Cm radicals that are linear, branched, or cyclic, and when cyclic, aromatic, or non-aromatic. Examples of such radicals include, but are not limited to, alkyl groups such as methyl, ethyl, n-propyl, isopropyl, n-butyl, isobutyl, sec-butyl, tert-butyl, pentyl, iso-amyl, hexyl, octyl cyclopropyl, cyclobutyl, cyclopentyl, cyclohexyl, cyclooctyl, and the like, aryl groups, such as phenyl, benzyl naphthyl, and the like.

Unless otherwise indicated, the term “substituted” means that at least one hydrogen atom has been replaced with at least one non-hydrogen group, such as a hydrocarbyl group, a heteroatom, or a heteroatom containing group, such as halogen (such as Br, Cl, F or I) or at least one functional group such as —NR*₂, —NR*—CO—R*, —OR*,*—O—CO—R*, —CO—O—R*, —SeR*, —TeR*, —PR*₂, —PO—(OR*)₂, —O—PO—(OR*)₂, —AsR*₂, —SbR*₂, —SR*, —SO₂—(OR*)₂, —BR*₂, —SiR*₃, —GeR*₃, —SnR*₃, —PbR*₃, —(CH₂)q-SiR*₃, or a combination thereof, where q is 1 to 10 and each R* is independently hydrogen, a hydrocarbyl or halocarbyl radical, and two or more R* may join together to form a substituted or unsubstituted completely saturated, partially unsaturated, or aromatic cyclic or polycyclic ring structure), or where at least one heteroatom has been inserted within a hydrocarbyl ring.

The term “substituted hydrocarbyl” means a hydrocarbyl radical in which at least one hydrogen atom of the hydrocarbyl radical has been substituted with at least one heteroatom (such as halogen, e.g., Br, Cl, F or I) or heteroatom-containing group (such as a functional group, e.g. —NR*₂, —NR*—CO—R*,—OR*,*—O—CO—R*, —CO—O—R*, —SeR*, —TeR*, —PR*₂, —PO— (OR*)₂, —O—PO—(OR*)₂, —AsR*₂, —SbR*₂, —SR*, —SO₂—(OR*)₂, —BR*₂, —SiR*₃, —GeR*₃, —SnR*₃, —PbR*₃, —(CH₂)q-SiR*₃, and the like, where q is 1 to 10 and each R* is independently hydrogen, a hydrocarbyl or halocarbyl radical, and two or more R* may join together to form a substituted or unsubstituted completely saturated, partially unsaturated, or aromatic cyclic or polycyclic ring structure), or where at least one heteroatom has been inserted within a hydrocarbyl ring.

The term “silyl group,” refers to a group comprising silicon atoms, such as a hydrosilylcarbyl group.

MALLS refers to multi angle laser light scattering and is sometimes referred to as MALS (multi angle light scattering).

As used herein, Mn is number average molecular weight, Mw is weight average molecular weight, and Mz is z average molecular weight, wt. % is weight percent, and mol % is mole percent. Molecular weight distribution (“MWD”), also referred to as polydispersity index (“PDI”), is defined to be Mw divided by Mn. Unless otherwise noted, all molecular weight units (e.g., Mw, Mn, Mz) are g/mol.

As used herein, room temperature is 23° C. unless otherwise indicated.

The terms “optional” or “optionally” means that the subsequently described event or circumstance may or may not occur, and that the description includes instances where the event or circumstance occurs and instances where it does not. For example, the phrase “optionally substituted hydrocarbyl” means that a hydrocarbyl moiety may or may not be substituted and that the description includes both unsubstituted hydrocarbyl and hydrocarbyl where there is substitution.

A “polymer” has two or more of the same or different mer units. A “homopolymer” is a polymer having mer units that are the same. A “copolymer” is a polymer having two or more mer units that are different from each other.

The term “ring atom” means an atom that is part of a cyclic ring structure.

A “ring carbon atom” is a carbon atom that is part of a cyclic ring structure.

The term “saturated” refers to the lack of double and triple bonds between atoms of a radical group such as ethyl, cyclohexyl, pyrrolidinyl, and the like. The term “unsaturated” refers to the presence of one or more double and triple bonds between atoms of a radical group such as vinyl, allyl, acetylide, oxazolinyl, cyclohexenyl, acetyl and the like, and specifically includes alkenyl and alkynyl groups, as well as groups in which double bonds are delocalized, as in aryl and heteroaryl groups as defined below.

The term “substituted” as in “substituted hydrocarbyl,” “substituted aryl,” “substituted alkyl,” and the like, means that in the group in question (i.e., the hydrocarbyl, alkyl, aryl or other moiety that follows the term), at least one hydrogen atom bound to a carbon atom is replaced with one or more substituent groups such as hydroxy, alkoxy, alkylthio, phosphino, amino, halo, silyl, and the like. When the term “substituted” introduces a list of possible substituted groups, it is intended that the term apply to every member of that group. That is, the phrase “substituted alkyl, alkenyl and alkynyl” is to be interpreted as “substituted alkyl, substituted alkenyl and substituted alkynyl.” Similarly, “optionally substituted alkyl, alkenyl and alkynyl” is to be interpreted as “optionally substituted alkyl, optionally substituted alkenyl and optionally substituted alkynyl.”

For compounds disclosed herein, any general or specific structure presented also encompasses all conformational isomers, regio-isomers, and stereoisomers that may arise from a particular set of substituents, unless stated otherwise. Similarly, the general or specific structure encompasses all enantiomers, diastereomers, and other optical isomers whether in enantiomeric or racemic forms, as well as mixtures of stereoisomers, unless stated otherwise.

It is to be understood that for purposes herein, when a radical is listed, it indicates the base structure of the radical (the radical type) and unless explicitly stated otherwise, all other radicals formed when that radical is subjected to substitution. Alkyl, alkenyl, and alkynyl radicals listed include all isomers including, where appropriate, cyclic isomers, for example, butyl includes n-butyl, 2-methylpropyl, 1-methylpropyl, tert-butyl, and cyclobutyl (and analogous substituted cyclopropyls); pentyl includes n-pentyl, cyclopentyl, 1-methylbutyl, 2-methylbutyl, 3-methylbutyl, 1-ethylpropyl, and nevopentyl (and analogous substituted cyclobutyls and cyclopropyls); butenyl includes E and Z forms of 1-butenyl, 2-butenyl, 3-butenyl, 1-methyl-1-propenyl, 1-methyl-2-propenyl, 2-methyl-1-propenyl, and 2-methyl-2-propenyl (and cyclobutenyls and cyclopropenyls). Cyclic compounds having substitutions include all isomer forms, for example, methylphenyl would include ortho-methylphenyl, meta-methylphenyl and para-methylphenyl; dimethylphenyl would include 2,3-dimethylphenyl, 2,4-dimethylphenyl, 2,5-dimethylphenyl, 2,6-diphenylmethyl, 3,4-dimethylphenyl, and 3,5-dimethylphenyl.

As referred to herein, the numbering schemes of the Periodic Table Groups are described in Chemical and Engineering News, vol. 63(5) at 27 (1985). A “Group 4 metal” is an element from group 4 of the Periodic Table and includes Hf, Ti, or Zr. A “Group 3 metal” is an element from Group 3 of the Periodic Table and includes lanthanum, scandium, or yttrium.

All numerical values within the detailed description herein are modified by “about” the indicated value, and include experimental error and variations that would be expected by a person having ordinary skill in the art.

### Isotactic and Syndiotactic Polymers

The physical properties of a polymer depend on its stereochemistry. Three different polymers are produced from racemic beta-butyrolactone: atactic, isotactic and syndiotactic as shown below:

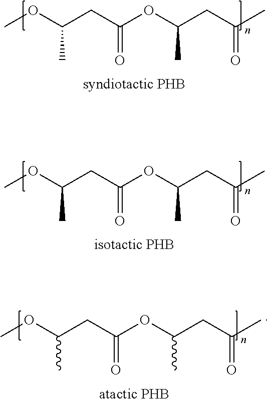

Atactic polymers can be synthesized with relatively simple catalyst systems. Due to their random nature, atactic polymers are typically amorphous and have low melting temperatures, making this type of polymer unsuitable for industrial applications.

Isotactic polymers are composed of isotactic macromolecules. In isotactic macromolecules, the substituents are located on the same side of the macromolecular backbone. An isotactic macromolecule consists of 100% meso diads, two identically oriented units. Isotactic polymers are typically semicrystalline and can form a helix configuration. Like many polymers, melting temperature of an isotactic polymer (including isotactic PHA) is close to its decomposition temperature, hindering commercial applications and processability. Furthermore, to produce an isotactic poly(3-hydroxybutyrate) from racemic β-butyrolactone is challenging.

In syndiotactic polymers, substituents have alternate positions along the backbone and is crystalline. This type of macromolecule consists 100% of racemo diads, units orientated in opposition. Syndiotactic polymers are crystalline. By increasing the number of regularly aligned its chains, hardness and density of the crystallized polymer are increased due to maximized intermolecular forces. Generally, syndiotactic polymers have a high melting temperature.

Depending on crystallinity, PHB can have similar tensile and mechanical properties to isotactic polypropylene. Other advantageous properties of PHB include solubility in water, chloroform and other chlorinated hydrocarbon, oxygen permeability, ultra-violet resistance, and less sticky when melted. PHB is biocompatible. The effect of stereoregularity on the thermal properties of PHB has been reported. Rieth, L. R. et al. (2002) “Single-Site β-Diiminate Zinc Catalysts for the Ring-Opening Polymerization of β-Butyrolactone and β-Valerolactone to Poly(3-hydroxyalkanoates),” J. Am. Chem. Soc., v.124(51), pp. 15239-15248. The melting temperature of isotactic PHB, especially PHB produced by an enzymatic process, is close to the decomposition temperature of the polymer hindering commercial application and processibility. However, the degree of syndiotacticity of PHB can impact the melting temperature of PHB. Therefore, as provided herein, the melting temperature (Tm) of PHB is tuned for better processability.

Currently, only a few methods have been described to produce syndiotactic polymers from racemic lactones. For example, tin-based catalysts were reported to produce atactic and syndiotactic-rich PHA polymers. Hori, Y. et al. (1999) “Ring-Opening Copolymerization of (R)-β-Butyrolactone with Macrolide: A New Series of Poly (Hydroxyalkanoate)s,” Macromolecules, v.32, pp. 3537-3539. These reactions are too slow (on the order of days to weeks) making them industrially irrelevant.

Also, aluminum, zinc, indium, and lanthanide-based catalyst systems to produce atactic, syndiotactic and isotactic PHB homopolymers and copolymers have been reported. Rieth, L. R. et al. (2002) “Single-Site β-Diiminate Zinc Catalysts for the Ring-Opening Polymerization of β-Butyrolactone and β-Valerolactone to Poly(3-hydroxyalkanoates),” J. Am. Chem. Soc., v.124(51), pp. 15239-15248. Ebrahimi, T. et al. (2016) “Highly Active Chiral Zinc Catalysts for Immortal Polymerization of β-Butyrolactone Form Melt Processable Syndio-Rich Poly(hydroxybutyrate),” Macromolecules, v.49(23), pp. 8812-8824. Lyubov, D. M. et al. (2019) “Rare-earth Metal Complexes as Catalysts for Ring-opening Polymerization of Cyclic Esters,” Coordination Chem. Rev., v.392, pp. 83-145. Shaik, M. et al. (2019) “Cyclic and Linear Polyhydroxylbutyrates from Ring-Opening Polymerization of β-Butyrolactone with Amino-Oxazolinate Zinc Catalysts,” Macromolecules, v.52(1), pp. 157-166. Sinenkov, M. A. et al. (2011) “Neodymium Borohydride Complexes Supported by Diamino-bis(phenoxide) Ligands: Diversity of Synthetic and Structural Chemistry, and Catalytic Activity in Ring-opening Polymerization of Cyclic Esters,” New J. Chem., v.35, pp. 204-212. Jaffredo, C. G. et al. (2013) “Poly(hydroxyalkanoate) Block or Random Copolymers of β-Butyrolactone and Benzyl β-Malolactone: A Matter of Catalytic Tuning,” Macromolecules, v.46, pp. 6765-6776. Ajellal, N. et al. (2009) “Syndiotactic-Enriched Poly (3-hydroxybutyrate)s via Stereoselective Ring-Opening Polymerization of Racemic β-Butyrolactone with Discrete Yttrium Catalysts,” Macromolecules, v.42, pp. 987-993. Altenbuchner, P. T. et al. (2015) “Mechanistic Investigations of the Stereoselective Rare Earth Metal-Mediated Ring-Opening Polymerization of β-Butyrolactone,” Chem. Eur. J., v.21, pp. 13609-13617. García-Valle, F. M., et al. (2018) “Biodegradable PHB from rac-β-Butyrolactone: Highly Controlled ROP Mediated by a Pentacoordinated Aluminum Complex,” Organometallics, v.37(6), pp. 837-840. Ebrahimi, T. et al. (2015) “Synthesis and Rheological Characterization of Star-Shaped and Linear Poly(hydroxybutyrate),” Macromolecules, v.48, pp. 6672-6681. Winnacker, M. (2019) “Polyhydroxyalkanoates: Recent Advances in Their Synthesis and Applications,” Eur. J. Lipid Sci. Technol., v.121, pp. 1-9.

Among the catalyst complexes, ONNO and ON(X)O type ligands shown immediately below have been investigated as possibly suitable for ROP of lactones, particularly BBL where the mechanism is reportedly chain-end controlled.

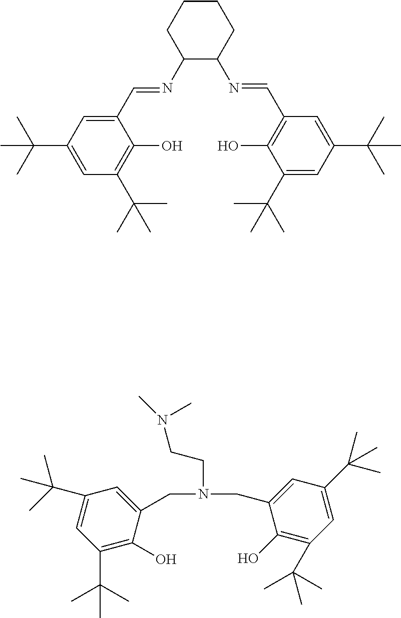

Li, H. et al. (2020) “Recent Advances in Metal-Mediated Stereoselective Ring-Opening Polymerization of Functional Cyclic Esters towards Well-Defined Poly(hydroxy acid)s: From Stereoselectivity to Sequence-Control,” Chem. Eur. J., v.26, pp. 128-138. Altenbuchner, P. T. et al. (2015) “Mechanistic Investigations of the Stereoselective Rare Earth Metal-Mediated Ring-Opening Polymerization of β-Butyrolactone,” Chem. Eur. J., v.21, pp. 13609-13617. Tschan, M. et al. (2021) “Controlling Polymer Stereochemistry in Ring-opening Polymerization: a Decade of Advances Shaping the Future of Biodegradable Polyesters,” Chem. Soc. Rev., v.50, pp. 13587-13608. Carpentier, J. (2010) “Discrete Metal Catalysts for Stereoselective Ring-Opening Polymerization of Chiral Racemic β-Lactones,” Macromol. Rapid Commun., v.31, pp. 1696-1705. Ajellal et al, supra, teaches microstructural and statistical analysis of syndiotactic PHBs produced by the ROP of rac-BBL with yttrium initiators.

The present catalyst systems differ from those previously reported for various reasons. First, the ligands described in the prior art are the type ON(X)O where X is a pendent amine or has ether functionality. See e.g., Ajellal, et al., supra. Second, ligands of the ONNO type are known to have a dimeric characteristic where an ether oxygen replaces a nitrogen. Carpentier, J. (2010) “Discrete Metal Catalysts for Stereoselective Ring-Opening Polymerization of Chiral Racemic β-Lactones,” Macromol. Rapid Commun., v.31, pp. 1696-1705. Third, ligands of the ONNO type often have imine or amine type functionalities in the backbone. Id; Fang, J. et al. (2013) “Yttrium Catalysts for Syndioselective β-butyrolactone Polymerization: on the Origin of Ligand-Induced Stereoselectivity,” Polym. Chem., v.4, pp. 360-367. Often the nitrogen atoms are linked with phenyl, cyclohexyl or ethylene bridges. The ligands provided herein take advantage of the oxophilicity of the early metals such yttrium by incorporating oxygen donors in the backbone. The hydrocarbon bridge between two oxygen atoms provides flexibility to the backbone.

ROP of rac-BLMe using the yttrium-salan complex Y{ONMeNMeOtBu2} was reported to generate highly syndiotactic PBHAs (Pr=0.90-0.94). Li, H. et al. (2020) “Recent Advances in Metal-Mediated Stereoselective Ring-Opening Polymerization of Functional Cyclic Esters Towards Well-Defined Poly(hydroxy acid)s: From Stereoselectivity to Sequence-Control,” Chem. Eur. J., v.26, pp. 128-138 citing Kramer, J. W. (2009) J. Am. Chem. Soc., v.131, pp. 16042-16044. Li also conducted initial studies on racemic β-butyrolactone which revealed that the Y{ON(X)OR′,R″} and Y{ONRNROtBu2} complexes can generate syndiotactic-rich PHB. Id. As reported, syndiotacticity increased with “steric bulkiness” of the ortho-R′ substituent on the {ON(X)OR′,R″} ligand framework. Id.

Similarly, this chemistry has been revisited by tuning the substituents on the N atoms of the salan ligand framework, and further using ytterbium (III) in addition to yttrium (III) complexes for the ROP of rac-BLMe. Zhuo, Z. et al. (2018) “Stereo-selectivity Switchable ROP of rac-β-butyrolactone Initiated by Salan-ligated Rare-Earth Metal Amide Complexes: the Key Role of the Substituents on Ligand Frameworks,” Chem. Commun., v.54, pp. 11998-12001. Independently of the nature of the metal center (that affects the activity), the tacticity of PHB was reportedly impacted by substituents on the bridging nitrogen atoms: cyclohexyl substituents, leading to syndiotactic-enriched PHB (Pr up to 0.78). They also reported that phenyl groups induce the formation of isotactic-enriched PHB (probability of meso linkage, Pm=1−Pr, up to 0.77), and that tert-butyl groups produce atactic PHB (Pm ca. 0.5). None of the reports, however, were focused on melt temperature. Further, no prior art catalyst system has been reported as producing PHB industrially. The focus has been on polymers having a high degree of syndiotacticity, but not on its processability. Yittrium is highly active and selective. As used in ring-opening polymerization, the degree of syndiotacticity of prior art catalyst systems has been shown to be widely variable.

Ring-opening polymerization conditions including the catalyst system must be matched to the type of polymer desired. In the case of PHB, the melting temperature as well as the decomposition temperature must be such that the polymer is later processed in various applications. For example, the challenges in processing PHB into flexible, thin films is a factor that prevent its widespread application. The high melting point of the PHB (˜175° C. to 180° C.) and low degradation temperature (˜220° C.) limit the possibility of thermal processing to prepare PHB films. Alternative approaches such as heat treatment, co-polymerization, blending and the addition of plasticizers have been used to improve the thermal processability. By using a combination of alternative approaches, PHB is extruded, rolled, and/or pressed into films having good mechanical properties. These alternative approaches, however, complicate processing and add costs.

As provided herein, catalyst systems comprising Group 3 metals are used in ring opening polymerization (ROP) of lactones to produce syndiotactic PHB having varying degree of syndiotacity and crystallinity. The present catalyst systems can produce unique microstructures for PHB and excellent mechanical and thermal properties. The present catalyst systems also produce syndiotactic PHB and allow for tuning the melting temperature of the PHB for better processability.

In the present ring-opening polymerization reaction of a β-lactone (i.e., β-butyrolactone), the metal precursor is dissolved in a suitable solvent and added to a catalyst solution of a ligand at room temperature. The catalyst solution is then stirred for three to five minutes. The catalyst solution is injected into a solution of the lactone and stirred. The general reaction is:

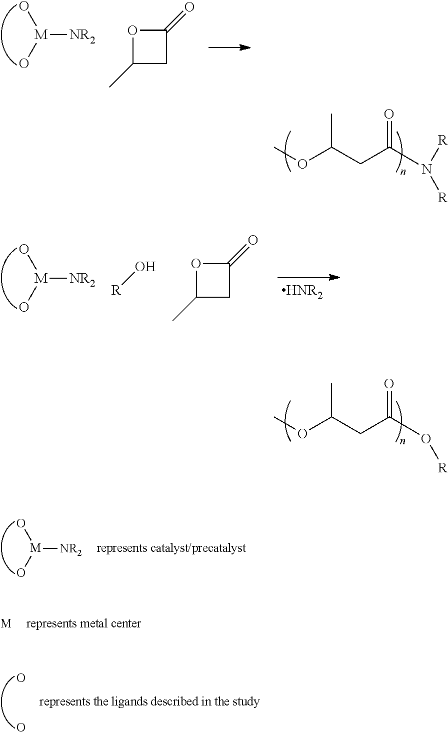

Polymer is isolated via precipitation and washed. The polymer is dried under vacuum. The tacticity of the polymer was determined by ¹³C NMR spectroscopy as described in Ebrahimi, T. et al., supra. The melting temperature of PHB was measured by Differential Scanning Calorimetry (DSC) is a routine technique to measure melting temperature, glass transition temperature and crystallinity of a polymeric sample. See generally, Hohne, G. W. H. et al. (2003) “Differential Scanning Calorimetry,” SpringerLink, pp. 115-146.

Other lactones polymerized using the present catalyst systems include:

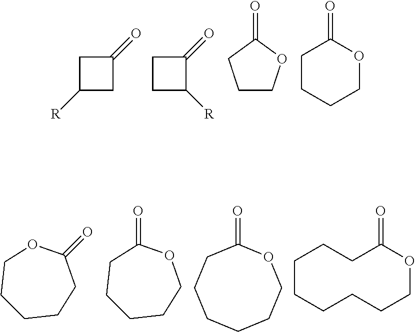

In the present in situ ring-opening polymerization processes, the metal precursor Y(CH₂SiCH₃)₃(THF)₂ has been found useful. However, it is not the only metal precursor that is used in these ring-opening polymerization processes. More generally, a metal precursor is YQ₃Sₙ, where Q is NR′₂, R″₃, or X₃, S is a Lewis base including solvent molecules such as nitrogen donors (amines, pyridine, nitrile groups etc.), oxygen donors (ethers such as diethyl ether, methyl tert-butyl ether, tetrahydrofuran, trialkylphosphine oxides etc.), phosphine donors (trialkyl phosphine such as tributylphosphine, triphenylphosphine etc.), sulfur donors such as thiophene, dimethyl sulfide etc., R′ is a hydrocarbyl group including Si groups, R″ is a hydrocarbyl group (including oxygen containing fragments) such as CH₂CMe₃, CH₂SiMe₃, CH₂C(CH₃)₂(C₆H₅), —CH₂Si(OCH₃)₃, n is 0 to 3, and X is F, Cl, Br, or I. Also, THF can be replaced by benzonitrile. See, Westerhausen, et al. (1995) Bis(trimethylsilyl)amide und-methanide des Yttriums-Molekülstrukturen von Tris(diethylether-O)lithium-(μ-chloro)-tris[bis(trimethylsilyl) methyl] yttriat, solvensfreiem Yttrium-tris[bis(trimethylsilyl)amid] sowie dem Bis(benzonitril)-Komplex,” Jrnl. Inorg. Chem., v.621(5), pp. 837-850. Diethyl ether can also replace THF. See., Eedugurala, N. et al. (2017) “β-SiH-Containing Tris(silazido) Rare-Earth Complexes as Homogeneous and Grafted Single-Site Catalyst Precursors for Hydroamination,” Organometalics, v.36(6), pp. 1142-1153.

### Ligands

As described herein, ligands used in the present catalyst systems include compounds having the structural formula

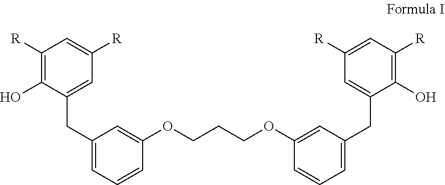

wherein R is independently selected from the group consisting of hydrogen, alkyl, substituted alkyl.

In an embodiment, the ligand of Formula I is a compound of the structural formula:

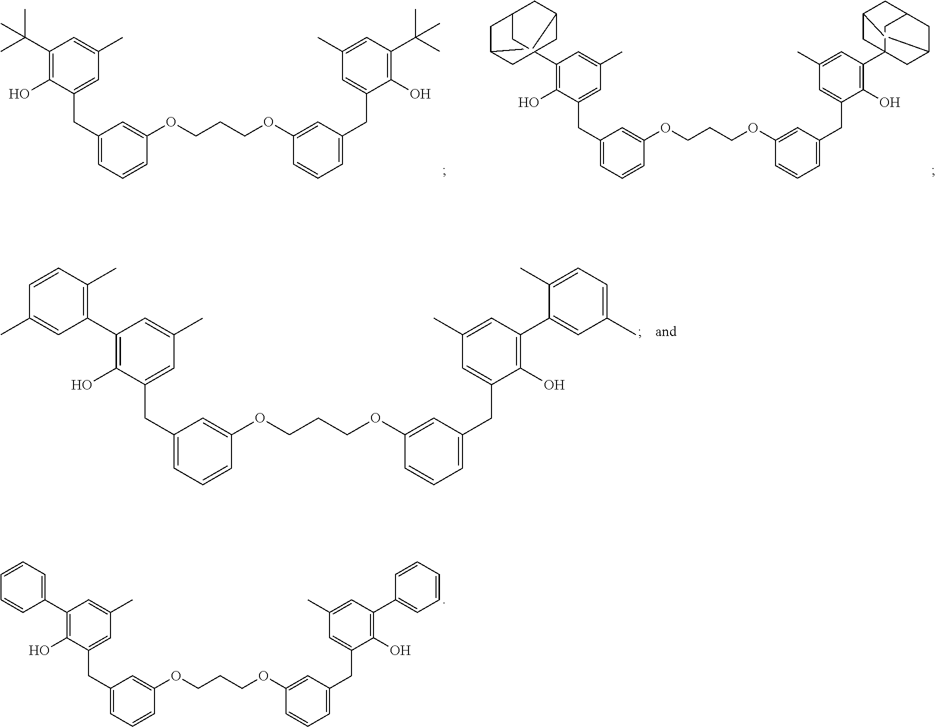

In an embodiment, the present catalyst systems can include a ligand having the structural formula

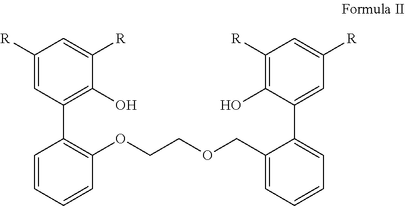

wherein R is independently selected from the group consisting of hydrogen, alkyl, substituted alkyl, aryl and substituted aryl.

The present catalyst system includes a ligand of the structural formula:

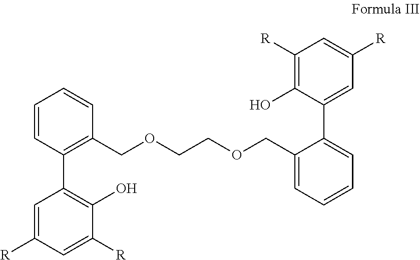

wherein R is independently selected from the group consisting of hydride, halide, optionally substituted hydrocarbyl, heteroatom-containing optionally substituted hydrocarbyl, alkoxy, aryloxy, silyl, boryl, alkyl, substituted alkyl, heteroalkyl, dialkyl amino, alkylthio, aryl and heteroaryl, arylthiol, and seleno, and optionally two or more R groups can combine together into a ring structure having 3 to 100 non-hydrogen atoms in the ring.

### General Synthesis—Ligands

Provided below is a general synthesis for making the ligand of Formula I is as follows:

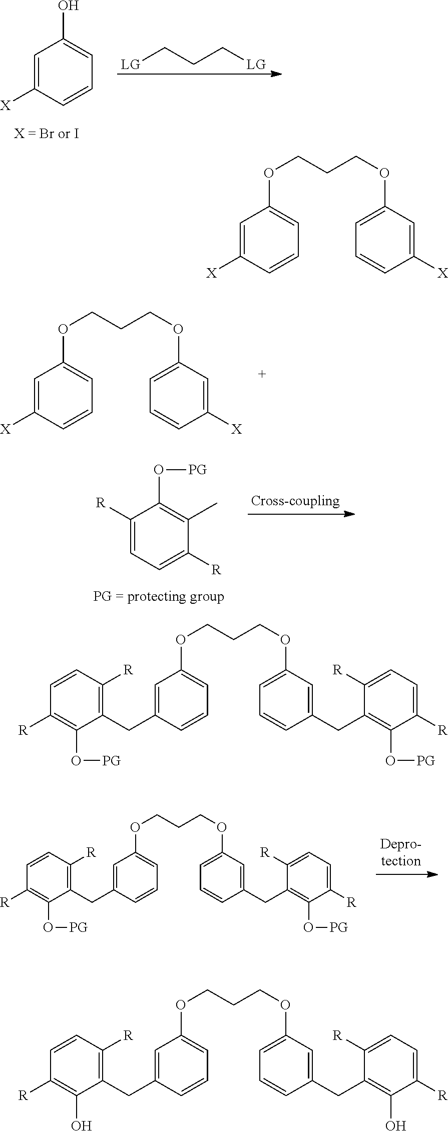

As shown above, the phenol dimer is synthesized by substitution of a phenol and a propyl linker. A cross-coupling of an ortho-methylphenol and a dimer yield the ligand following the deprotection of the phenol groups.

The general synthesis for making a compound of the Formula II is as follows:

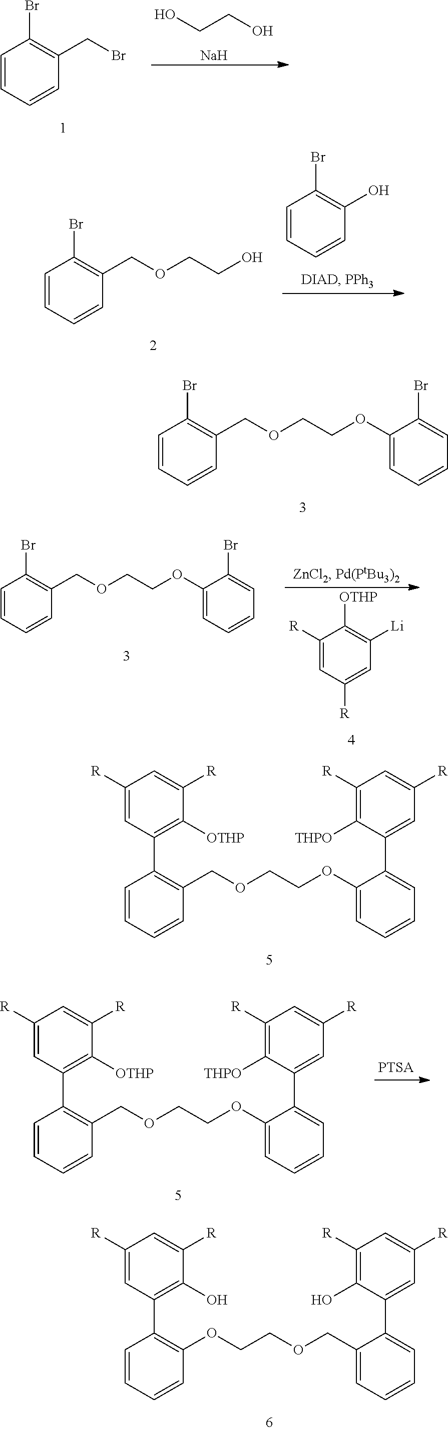

The coupling of ethylene glycol and the substituted bromobenzene 1 yielded the ether 2 which was then reacted with ortho-bromophenol to form the intermediate 3. Following the addition of the substituted phenyl lithium species 4 to 3, the biaryl intermediate 5 was deprotected to generate the ligand 6.

The general synthesis for making a ligand of the Formula III is as follows:

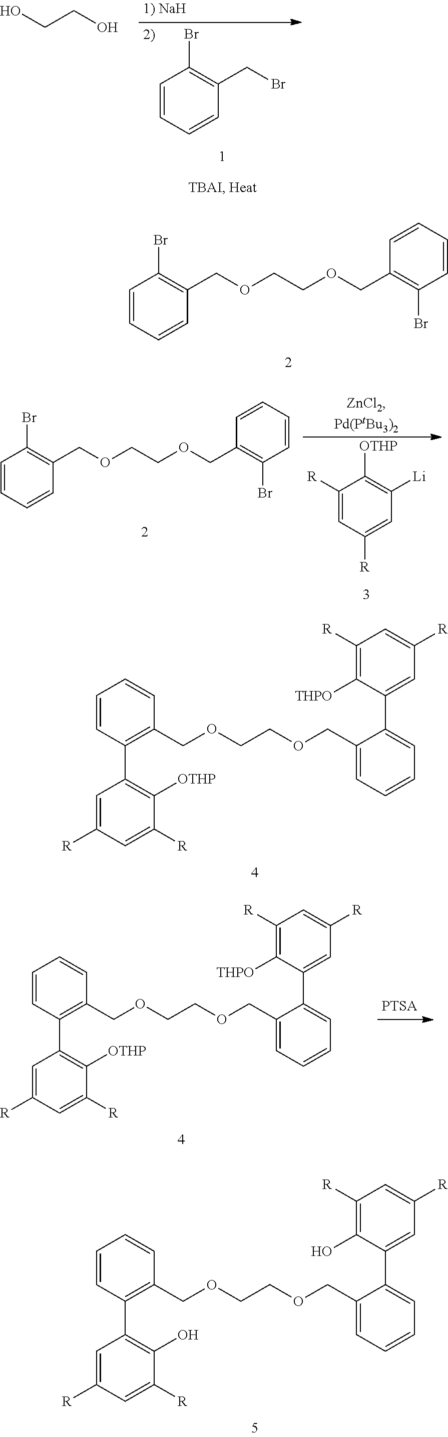

The coupling of ethylene glycol and the substituted bromobenzene 1 generated the intermediate 2. Following the addition of the substituted phenyl lithium species 3 to 2, the biaryl intermediate 4 was deprotected to generate the ligand 5.

### Catalyst Systems—In Situ

As described in the examples, the catalyst systems are used in in situ ring-opening polymerization. In the present methods, ring-opening polymerization using the present catalyst systems is conducted at room temperature (23° C.).

In situ, the catalyst systems comprise a combination of one or more of the present ligands and a metal atom, ion, or compound, or metal precursor. For example, the ligand is added to a reaction vessel at the same time as the metal or metal precursor compound along with lactone, reactants, activators, scavengers, etc. Additionally, the ligand is modified prior to addition to or after the addition of the metal precursor, e.g., through a deprotonation reaction or some other modification.

In general, a metal precursor can be characterized by the general formula M(L)ₘ where M is a Group 3 metal and m is 1, 2, 3, 4, 5, or 6. Each L is a ligand as defined herein. Optionally, two or more L groups are joined into a ring structure. One or more of the ligands L are ionically bonded to the metal M and, for example, L may be a non-coordinated or loosely coordinated or weakly coordinated anion (e.g., L is selected from the group consisting of those anions described below in the conjunction with the activators). See Marks et al. (2000) Chem. Rev., v.100, pp. 1391-1434, for a detailed discussion of the weak interactions. The metal precursors are monomeric, dimeric, or higher orders thereof. Metal precursors include the metal yttrium and other Group 3 metals such as Sc, La and Ac.

The ligand to metal precursor compound ratio is typically in the range of about 0.01:1 to about 100:1, about 0.1:1 to about 10:1 and about 1:1, 2:1 or 3:1. An activator (also referred to as a “co-catalyst”) can also be used in the polymerization methods described herein. The activator “activates” a catalyst complex by converting the neutral catalyst compound to a catalytically active catalyst complex. Such activators can include trialkyl aluminum compounds: trimethylaluminum, triethylaluminum, triisobutylaluminum, tri-n-hexylaluminum, tri-n-octylaluminu and alumoxanes including methyl alumoxane (MAO), modified methylalumoxane (MMAO), ethylalumoxane, isobutylalumoxane, and solid polymethylaluminoxane.

Non-coordinating anions (NCA) can also be the activator in the methods described herein including, for example, N,N-dimethylanilinium tetrakis(pentafluorophenyl)borate, tris(pentafluorophenyl)boron, tri(n-butyl) ammonium tetrakis(pentafluorophenyl)borate, a tris perfluorophenyl boron metalloid precursor or a tris perfluoronaphthyl boron metalloid precursor, polyhalogenated heteroborane anions, boric acid, or combination thereof.

### Additional Embodiments

Embodiment 1. A catalyst system useful in ring-opening polymerization of lactones comprising a metal precursor comprising a Group 3 metal and a ligand of the Formula I:

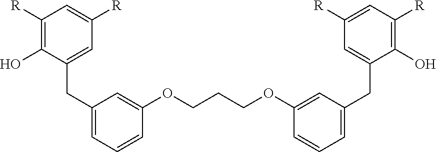

wherein R is selected from the group of hydrogen, alkyl and substituted alkyl.

Embodiment 2. The catalyst system of Embodiment 1, wherein the Group 3 metal precursor comprises yttrium, scandium, and/or lanthanum.

Embodiment 3. The catalyst system of Embodiment 1, wherein the metal precursor is Y[N(SiHMe2)]₃(THF)₂.

Embodiment 4. The catalyst system of Embodiment 1, 2, or 3 wherein the ligand of Formula I is:

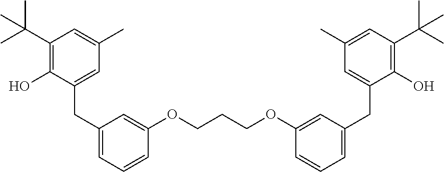 [SMILES: Cc1cc(Cc2cccc(OCCCOc3cccc(Cc4cc(C)cc(C(C)(C)C)c4O)c3)c2)c(O)c(C(C)(C)C)c1]

Embodiment 5. The catalyst system of Embodiment 1, 2, or 3 wherein the ligand of Formula I is:

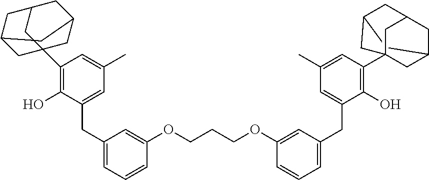 [SMILES: Cc1cc(Cc2cccc(OCCCOc3cccc(Cc4cc(C)cc(C56CC7CC(CC(C7)C5)C6)c4O)c3)c2)c(O)c(C23CC4CC(CC(C4)C2)C3)c1]

Embodiment 6. The catalyst system of Embodiment 1, 2, or 3 wherein the ligand of Formula I is:

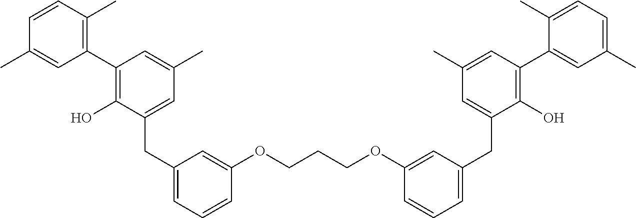

Embodiment 7. The catalyst system of Embodiment 1, 2, or 3 wherein the ligand of Formula I is:

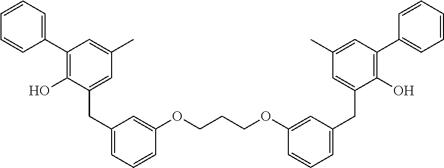 [SMILES: Cc1cc(Cc2cccc(OCCCOc3cccc(Cc4cc(C)cc(-c5ccccc5)c4O)c3)c2)c(O)c(-c2ccccc2)c1]

Embodiment 8. A method of ring-opening polymerization of cyclic ethers and esters comprising mixing a catalyst system comprising a Group 3 metal precursor and a ligand of the structural Formula I:

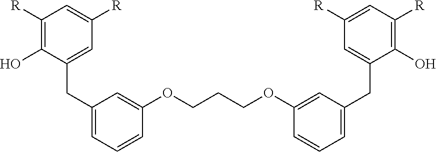

with one or more lactones at room temperature to provide a syndiotactic polymer, wherein R is selected from group of hydrogen, alkyl and substituted alkyl.

Embodiment 9. The method of ring-opening polymerization of cyclic ethers and esters of Embodiment 8, wherein the cyclic ethers and esters comprise β-butyrolactone.

Embodiment 10. The method of ring-opening polymerization of cyclic ethers and esters of Embodiment 8, wherein the syndiotactic polymer is PHB.

Embodiment 11. The method of ring-opening polymerization of cyclic ethers and esters of Embodiment 8, 9, or 10 wherein the syndiotactic polymer has a Tm of between 85° C. and 110° C.

Embodiment 12. The method of ring-opening polymerization of cyclic ethers and esters of Embodiment 8, 9, 10, or, 11 wherein the syndiotactic polymer has a Pr of between .55 and .70.

Embodiment 13. A method of in situ ring-opening polymerization of β-butyrolactone comprising the steps of:
adding a metal precursor comprising a Group 3 metal in a solvent to a catalyst solution comprising a ligand of the structural Formula I;
adding β-butyrolactone to the catalyst solution; and
mixing the catalyst solution for at least 3 minutes; and
separating PHB from the catalyst solution, wherein the PHB has a Pr of at least .55.

Embodiment 14. The method in situ ring-opening polymerization of β-butyrolactone of Embodiment 13, wherein the ligand is a compound selected from the group:

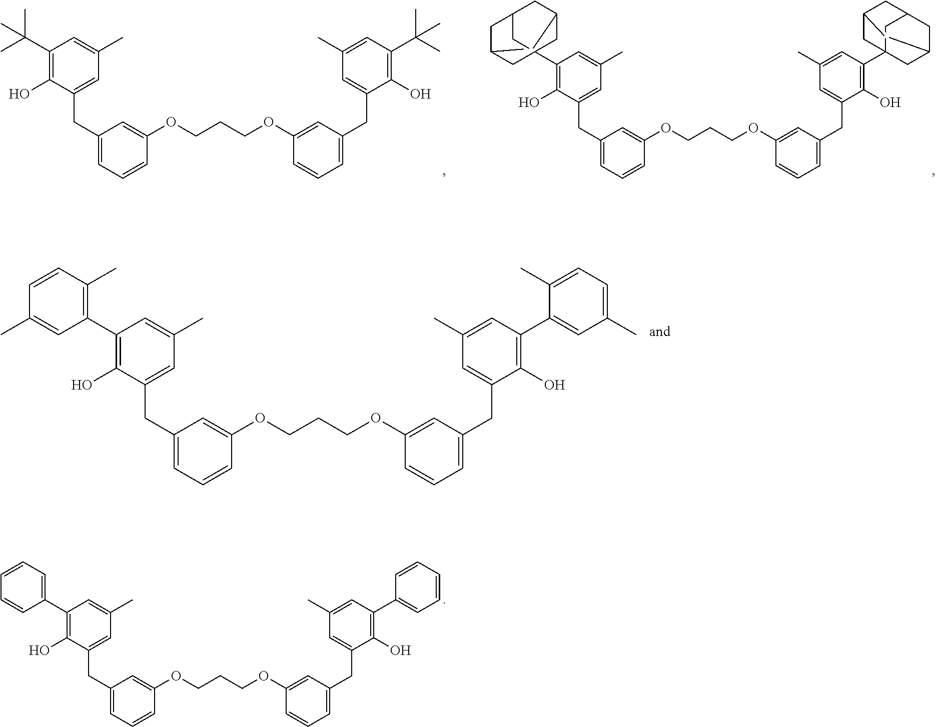

Embodiment 15. The method of Embodiment 13 or 14, wherein the ring-opening polymerization is conducted at 23° C.

Embodiment 16. The method of Embodiment 13, 14, or 15, wherein the PHB has a Tm between 85° and 110° C.

Embodiment 17. A compound of structural Formula I

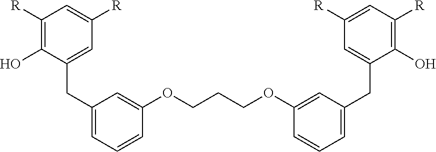

wherein R is selected from group of hydrogen, alkyl and substituted alkyl.

Embodiment 18. An isolated yttrium complex comprising yttrium and a ligand comprising a compound of the structural Formula I

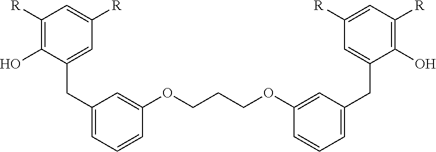

Wherein R is selected from the group of hydrogen, alkyl and substituted alkyl.

Embodiment 19. A catalyst system comprising the isolated yttrium complex of Embodiment 18.

Embodiment 20. The catalyst system of Embodiment 19 wherein polymerization is initiated through an alcohol and amine exchange.

The following non-limiting examples are provided to illustrate the disclosure.

### EXAMPLES

### Example 1

### Ring-Opening Polymerization Using the Present Ligands

1 g toluene solution of Y[N(SiHMe₂)₂]₃(THF)₂was added to a 1.5 g toluene solution of each ligand (Entry 1, Entry 2, Entry 3, and Entry 4) at room temperature to provide a catalyst solution. The catalyst solution was stirred for three (3) minutes. β-butyrolactone was then added to the stirring catalyst solution and left stirring at this temperature for the specified amount of time. Once the reaction was complete, the reaction mixture was poured onto excess methanol or isopropanol and the precipitate was isolated by decanting the reaction mixture. Precipitated polymer was then dried in a vacuum oven until constant weight.

Tables 1A & 1B immediately below provide the details of each experiment where the reaction temperature was at room temperature.

TABLE 1A

| | | Amount | | | |
|---|---|---|---|---|---|
| | Amount | pre- | | | BBL: |
| En- | ligand | cursor | | Metal | cata- |
| try | (mg) | (mg) | Ligand structure | precursor | lyst |
| 1 | 9 | 9.7 | 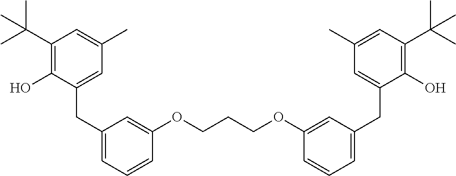 [SMILES: Cc1cc(Cc2cccc(OCCCOc3cccc(Cc4cc(C)cc(C(C)(C)C)c4O)c3)c2)c(O)c(C(C)(C)C)c1] | Y[N(SiHMe₂)₂]₃(THF)₂ | 1000: 1 |
| 2 | 10 | 8.5 | 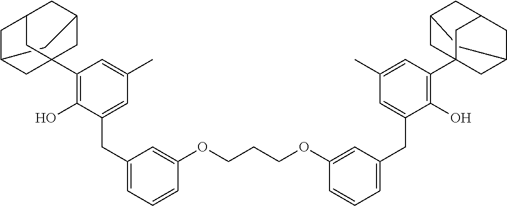 [SMILES: Cc1cc(Cc2cccc(OCCCOc3cccc(Cc4cc(C)cc(C56CC7CC(CC(C7)C5)C6)c4O)c3)c2)c(O)c(C23CC4CC(CC(C4)C2)C3)c1] | Y[N(SiHMe₂)₂]₃(THF)₂ | 1000: 1 |
| 3 | 10 | 9.3 | 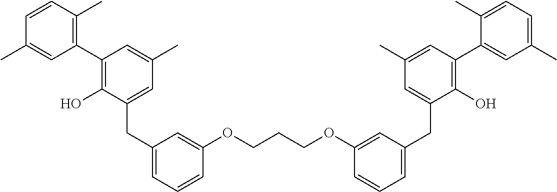 | Y[N(SiHMe₂)₂]₃(THF)₂ | 1000: 1 |
| 4 | 10 | 10.1 | 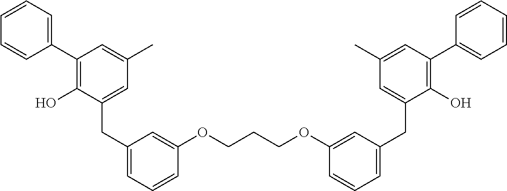 [SMILES: Cc1cc(Cc2cccc(OCCCOc3cccc(Cc4cc(C)cc(-c5ccccc5)c4O)c3)c2)c(O)c(-c2ccccc2)c1] | Y[N(SiHMe₂)₂]₃(THF)₂ | 1000: 1 |

TABLE 1B

| | | | Mw | Mn | | | | yield |
|---|---|---|---|---|---|---|---|---|
| Entry | T | t(h) | (DRI) | (DRI) | PDI | Pr | Tm(° C.) | (isolated) |
| 1 | rt | 16 | 82,385 | 67,341 | 1.22 | 0.55 | 102.93 | 35% |
| 2 | rt | 16 | 98,509 | 53,609 | 1.84 | 0.53 | 108.75 | 47% |
| 3 | rt | 24 | 20,907 | 14,247 | 1.47 | 0.59 | Not | 30% |
| | | | | | | | measured | |
| 4 | Rt | 24 | 48,983 | 31,479 | 1.56 | 0.69 | 89.39 | 60% |

For Entry 1 and Entry 2 A 30° pulse, 2 s delay, 4096 transients, and gated decoupling were used for measuring the ¹³C NMR. For Entry 3 and Entry 4 A 30° pulse, 2 s delay, 2048 transients, and gated decoupling were used for measuring the ¹³C NMR. Assignments and calculation of tacticity were made in accordance with teaching of Ajellal, N et. al., supra, Altenbuchner, P.T. et al., supra, and Ebrahimi, T. et al., supra.

The molecular weight moments (M_{w}, Mₙ) and distribution (“PDI”) for the polymers were determined by gel permeation chromatography (“GPC”). The chromatographic system included an HPLC pumping system equipped with solvent degasser, autosampler (Agilent 1100), three 10-μm Mixed-B-LS columns (Agilent, PLgel), a light scattering detector (Wyatt DAWN® Heleos II) and a differential refractive index detector (Wyatt Optilab® T-rEX). The GPC system was operated at room temperature with chloroform mobile phase (Aldrich, HPLC grade) at flow rate of 1.0 ml/min and injection volume of 100 uL. The polymer samples were dissolved in chloroform at approximately 3 milligram per milliliter (mg/ml at room temperature. The solutions were filtered through a 0.2 micron PTFE filter prior to injection. The dn/dc value of 0.03 used in the light scattering analysis was an average value determined from the DRI detector by assuming 100% mass recovery and was used for the light scattering analysis of all PHB samples.

Samples were dissolved in deuterated chloroform (CDCl₃) at a concentration of 20-80 mg/mL between 25° C. and 30° C. Spectra were recorded at between 25° C. and 30° C. using a Bruker NMR spectrometer of at least 500 MHz with a 5 mm broadband probe.

### Experimental Results

The results provided above show that the catalyst systems prepared in situ with the present bisphenol ligands have good activity and selectivity towards syndiotactic PHB polymers having a Tm as high as 109° C. While there were previous reports of a nearly linear relationship between Pr and Tm in the resulting polymer produced by ON(X) type ligands, the linear relationship does not exist when the present ligands were used in the catalyst systems tested in this study.

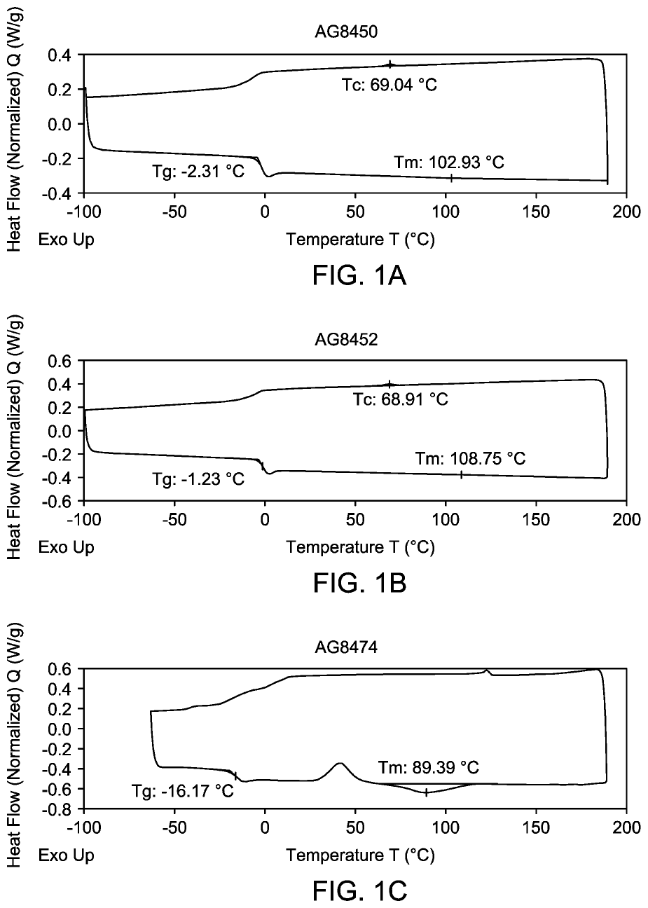

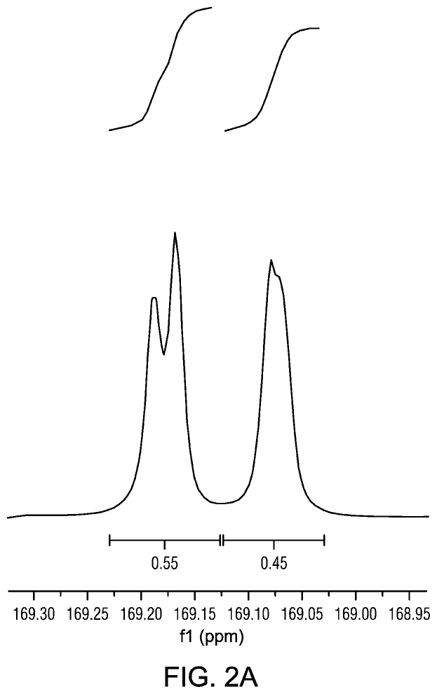

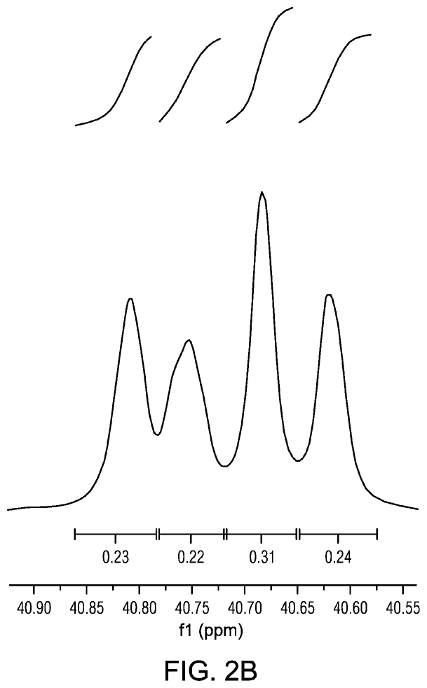

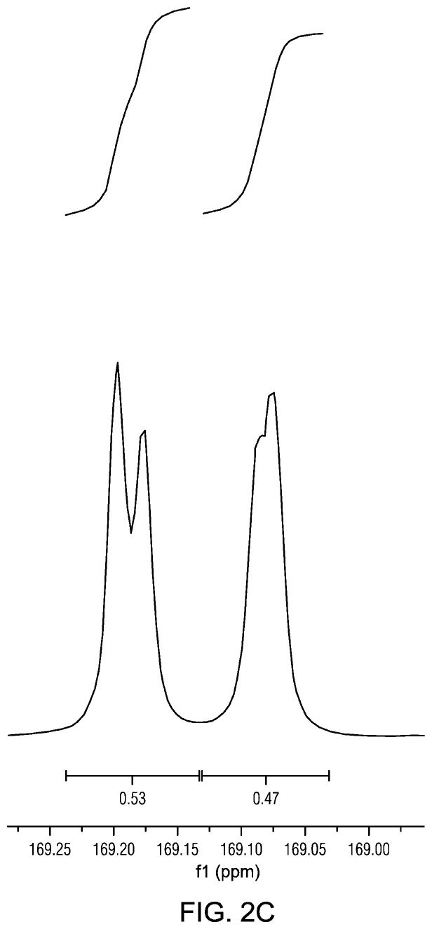

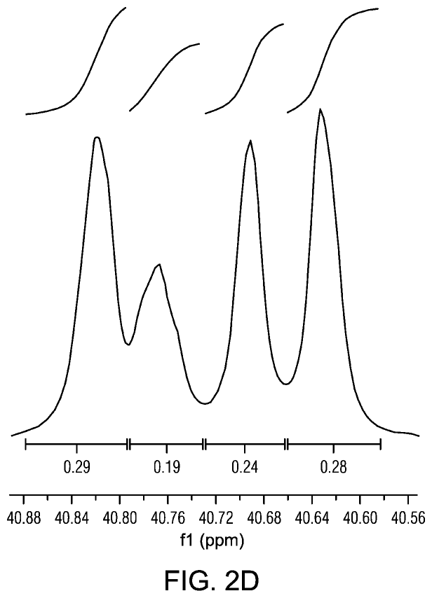

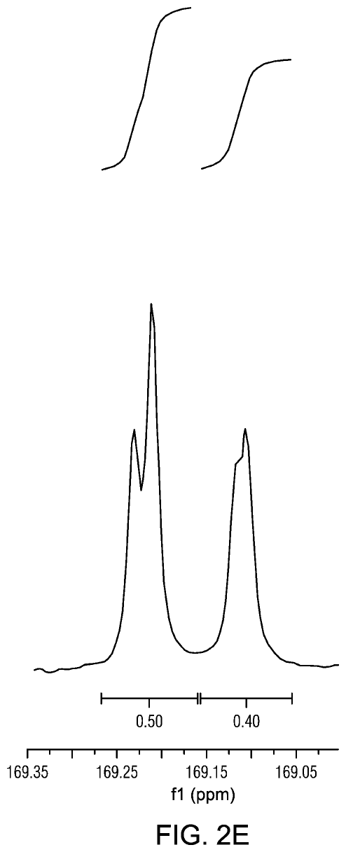

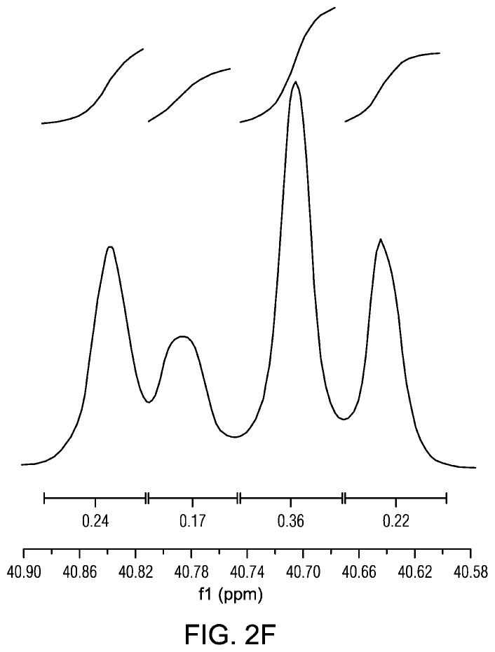

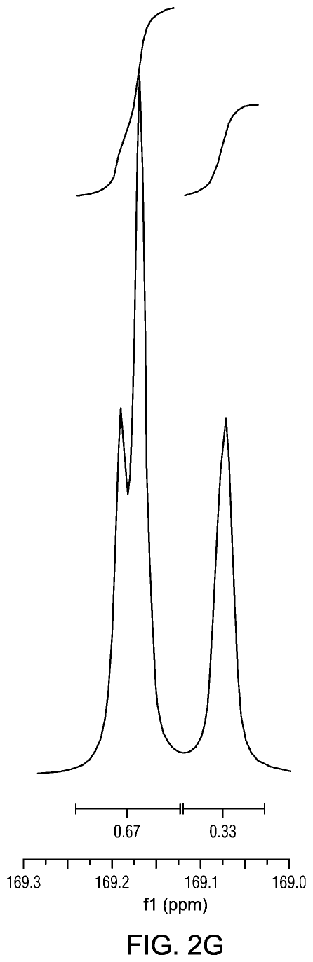

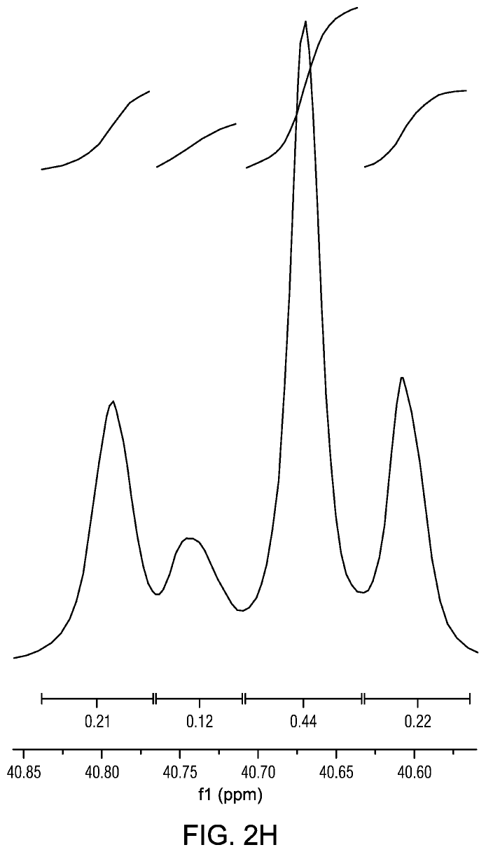

For example, PHB having a Pr of 0.53 or 0.55 is not expected to have a melting temperature. However, polymers in Entry 1 and Entry 2 both have a melting temperature and to some degree even crystallinity temperature (“Tc”). FIG. 1A & FIG. 1B. Similarly, in Entry 4 the Pr is 0.69 indicating that highly syndio-enriched PHB which provide a higher Tm when compared to that of Entry 1 or Entry 2. FIG. 1C. DSC measurements indicate that the polymer has a lower Tm. FIG. 2A, FIG. 2B, FIG. 2C, FIG. 2D, FIG. 2E, FIG. 2F, FIG. 2G and FIG. 2H. A racemic linkage, Pr, is 1.0 for a perfectly syndiotactic polymer. For an atactic polymer, Pr is approximately 0.5 as measured by ¹³C NMR. The Pr for the present syndiotactic PHB is .55 or higher.

Also as evident by data presented in Tables 1A and 1B, syndiotacticity is changed or tuned by simply changing ortho substituent to the phenol —OH. More electron donating alkyl substituents such as t-butyl (Entry 1 and adamantane (Entry 2) substitutions result in higher melting polymers. More electron withdrawing substituents such as aromatic phenyl groups (Entry 4) produce polymers with lower melting temperatures.

Moreover, electron donating ligands (Entry 1 and 2) also resulted in higher molecular weight polymers indicating that simple electronic effects of the ligands could change MW capabilities of the catalyst. PHB having high melting temperature, however, is not beneficial from the product and process point of view because the polymer decomposes above its melting point. Therefore, the polymer is not suitable for process - even though PHB polymer has various advantages. PHB has numerous problems especially from a process standpoint. For example, biogenic PHB (produced by enzymes) has a high Tm (˜180° C.). But it degrades around 220° C. making it difficult to process. Therefore, to be able to tune the Tm, particularly to lower it to a temperature that processing is industrially achievable is valuable. The present catalyst systems described herein show that access to lower melting temperatures is possible with the present ligands.

DSC data shows a difference in the crystallization and melting temperatures of the syndiotactic polymers produced between 35 and 40° C. FIG. 2A, FIG. 2B, FIG. 2C, FIG. 2D, FIG. 2E, FIG. 2F, FIG. 2G and FIG. 2H.

Tacticity was measured by following earlier described methods. Pr as provided in the Tables are calculated by integrating the carbonyl region of 13C NMR spectra of the isolated polymers. In this case, Pr value represents the syndiotacticity of the polymer microstructure where r means the probability of racemic linkages between monomer units. Methylene carbon regions are also provided as supplemental information. Pr values are also calculated based on the methylene regions as described in literature and they are in perfect agreement with the Pr values measured from the carbonyl region. Entry 1 has a Pr equal to .55. Entry 2 has a Pr equal to .53. Entry 3 has a Pr equal to 60. Entry 4 has a Pr of 0.67.

### Example 2

### Synthesis of Isolated Yttrium Complex

To a 0.5 g benzene solution of Y[N(SiHMe₂)₂]₃(THF)₂ (40 mg), 0.5 g benzene solution of ligand (37 mg) was added at room temperature and left stirring for 16 hours. Solvent was removed in vacuum and the remaining solids were washed with 2 mL pentane and 2 mL diethyl ether. The white solids were dried in vacuum to constant weight (40 mg). The ligand used in this experiment was:

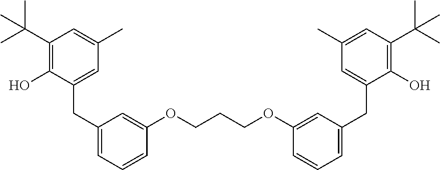 [SMILES: Cc1cc(Cc2cccc(OCCCOc3cccc(Cc4cc(C)cc(C(C)(C)C)c4O)c3)c2)c(O)c(C(C)(C)C)c1]

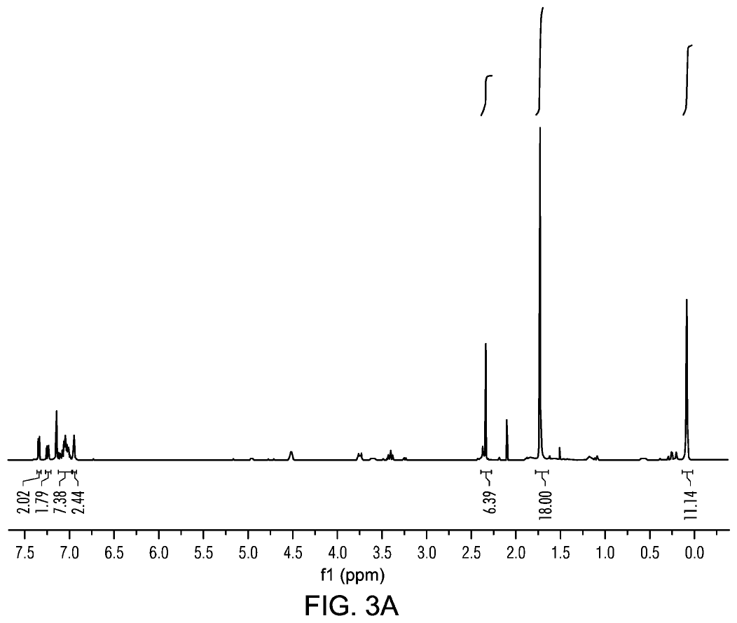

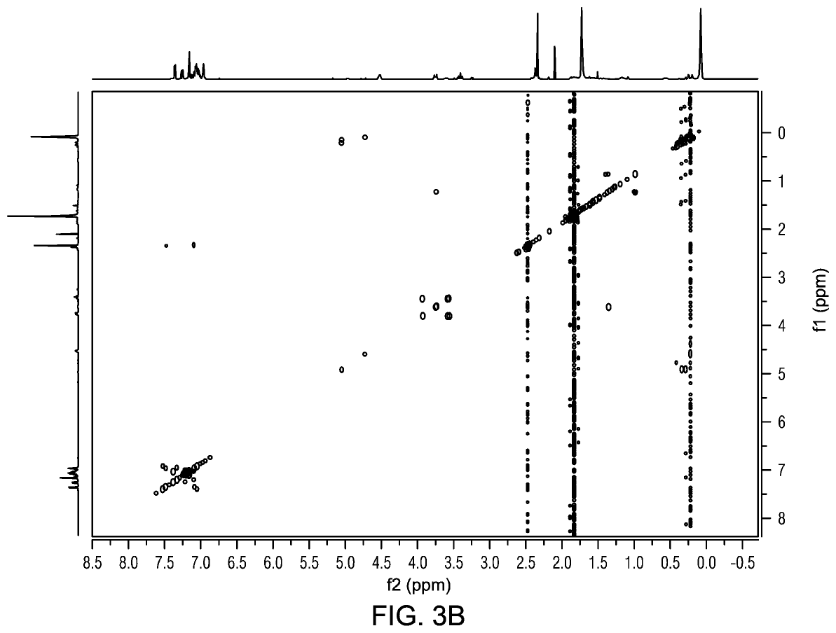

The ¹H NMR of the isolated complex exhibits chemical shifts and integration for the t-butyl, and —CH₃ groups both on the ligand and the —N(SiHMe₂)₂ group. FIG. 3A. These peaks are assigned at 2.32 ppm (6H), 1.73 ppm (18H) and 0.08 ppm (12H). The aromatic region also contains the expected 12H between the region from 6.95 ppm to 7.35 ppm. NMR studies revealed that assigning the —CH₂ groups has been challenging due to the likely fluxional behavior of the ligand. 2D COSY NMR experiment was used to further assign these peaks, but assignment was not possible. FIG. 3B. Tetradentate ligands can exhibit this kind of behavior in solution as shown immediately below.

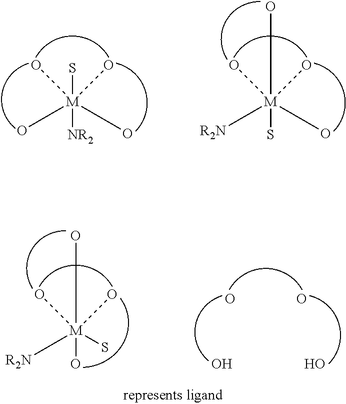

S=Lewis Base, THF, diethyl ether, pyridine, PR₃, NR₃ and the like.
M=Yttrium and/or lanthanide metal ion

### Example 3

### Polymerization Using the Isolated Yttrium Complex

40 mg of the complex isolated according to Example 2 above was dissolved in 2 mL C₆D₆ and neat lactone (1.51 g) was added to the stirring solution at room temperature. The solution was then stirred at room temperature for 2 hours after which the stirring came to a complete stop due to viscosity. The gel was dissolved in excess chloroform and precipitated with excess methanol. The white solids were isolated and dried in a vacuum oven to constant weight. 1.35 gram. The resulting polymer had a syndio-enriched microstructure with a Pr of 0.67 and a Tm of 108° C. based on the DSC.

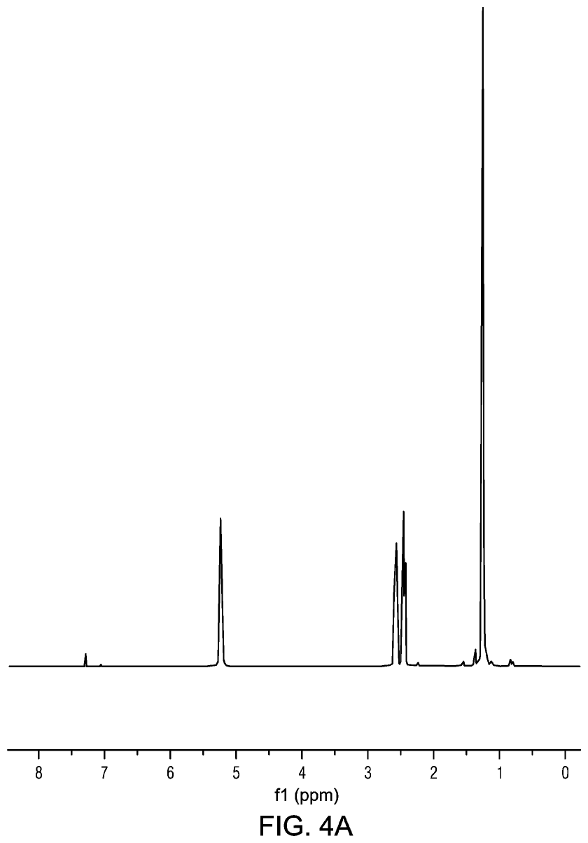

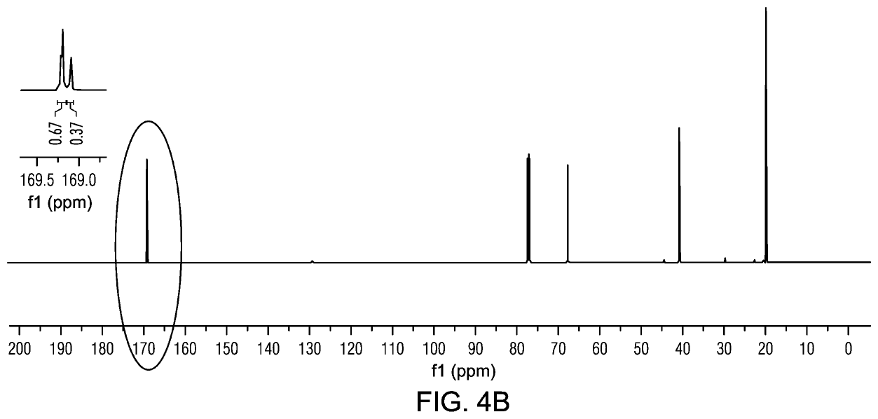

Based on the comparison of the preceding Examples (in situ polymerization and polymerization using the isolated complex), isolation of the metal complex led to higher stereoselectivity in the polymerization process as observed by both a higher Pr (¹³C NMR) and a higher Tm (DSC) of the resulting polymer. FIG. 4A & FIG. 4B. This represents an advantage of the ligand-metal combinations described herein such that microstructure is further controlled by the manner which the catalyst system is generated.

Many alterations, modifications, and variations will be apparent to those skilled in the art in light of the foregoing description without departing from the spirit or scope of the present disclosure and that when numerical lower limits and numerical upper limits are listed herein, ranges from any lower limit to any upper limit are contemplated.

We claim:

1. A catalyst system useful in ring-opening polymerization of lactones comprising a metal precursor comprising a Group 3 metal and a ligand of the Formula I: 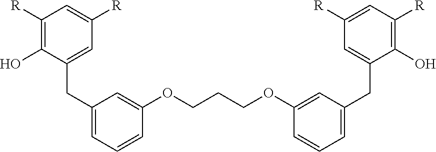
wherein R is selected from the group of hydrogen, alkyl and substituted alkyl.

2. The catalyst system of claim 1, wherein the Group 3 metal precursor comprises yttrium, scandium, and/or lanthanum.

3. The catalyst system of claim 1, wherein the metal precursor is Y[N(SiHMe2)]₃(THF)₂.

4. The catalyst system of claim 1, wherein the ligand of Formula I is:

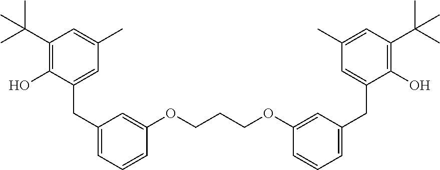 [SMILES: Cc1cc(Cc2cccc(OCCCOc3cccc(Cc4cc(C)cc(C(C)(C)C)c4O)c3)c2)c(O)c(C(C)(C)C)c1]

5. The catalyst system of claim 1, wherein the ligand of Formula I is:

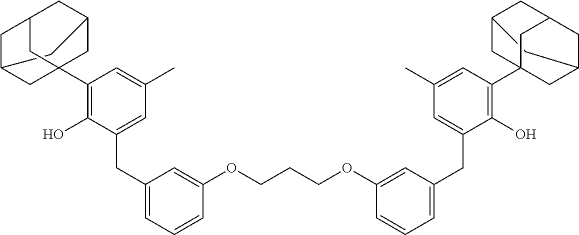 [SMILES: Cc1cc(Cc2cccc(OCCCOc3cccc(Cc4cc(C)cc(C56CC7CC(CC(C7)C5)C6)c4O)c3)c2)c(O)c(C23CC4CC(CC(C4)C2)C3)c1]

6. The catalyst system of claim 1, wherein the ligand of Formula I is:

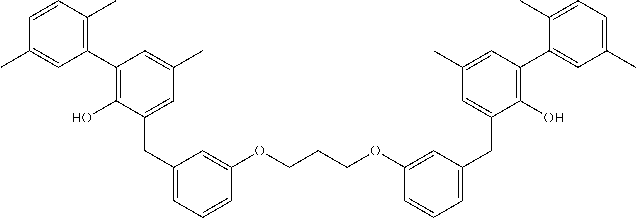

7. The catalyst system of claim 1, wherein the ligand of Formula I is:

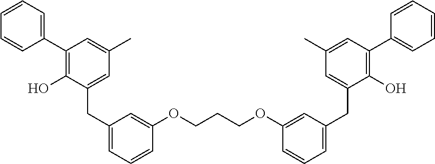 [SMILES: Cc1cc(Cc2cccc(OCCCOc3cccc(Cc4cc(C)cc(-c5ccccc5)c4O)c3)c2)c(O)c(-c2ccccc2)c1]

8. A method of ring-opening polymerization of cyclic ethers and esters comprising mixing a catalyst system comprising a Group 3 metal precursor and a ligand of the structural Formula I 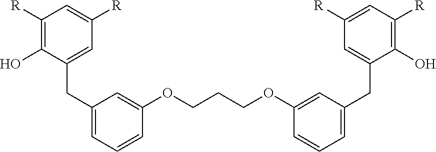
with one or more lactones at room temperature to provide a syndiotactic polymer, wherein R is selected from the group of hydrogen, alkyl and substituted alkyl.

9. The method of ring-opening polymerization of cyclic ethers and esters of claim 8, wherein the cyclic ethers and esters comprise β-butyrolactone.

10. The method of ring-opening polymerization of cyclic ethers and esters of claim 8, wherein the syndiotactic polymer is PHB.

11. The method of ring-opening polymerization of cyclic ethers and esters of claim 8, wherein the syndiotactic polymer has a Tm of between 85° C. and 110° C.

12. The method of ring-opening polymerization of cyclic ethers and esters of claim 8, wherein the syndiotactic polymer has a Pr of between .55 and .70.

13. A method of in situ ring-opening polymerization of β-butyrolactone comprising the steps of:
adding a metal precursor comprising a Group 3 metal in a solvent to a catalyst solution comprising a ligand of the structural Formula I;
adding β-butyrolactone to the catalyst solution; and
mixing the catalyst solution for at least 3 minutes; and
separating PHB from the catalyst solution, wherein the PHB has a Pr of at least .55.

14. The method in situ ring-opening polymerization of β-butyrolactone of claim 13, wherein the ligand is a compound selected from the group:

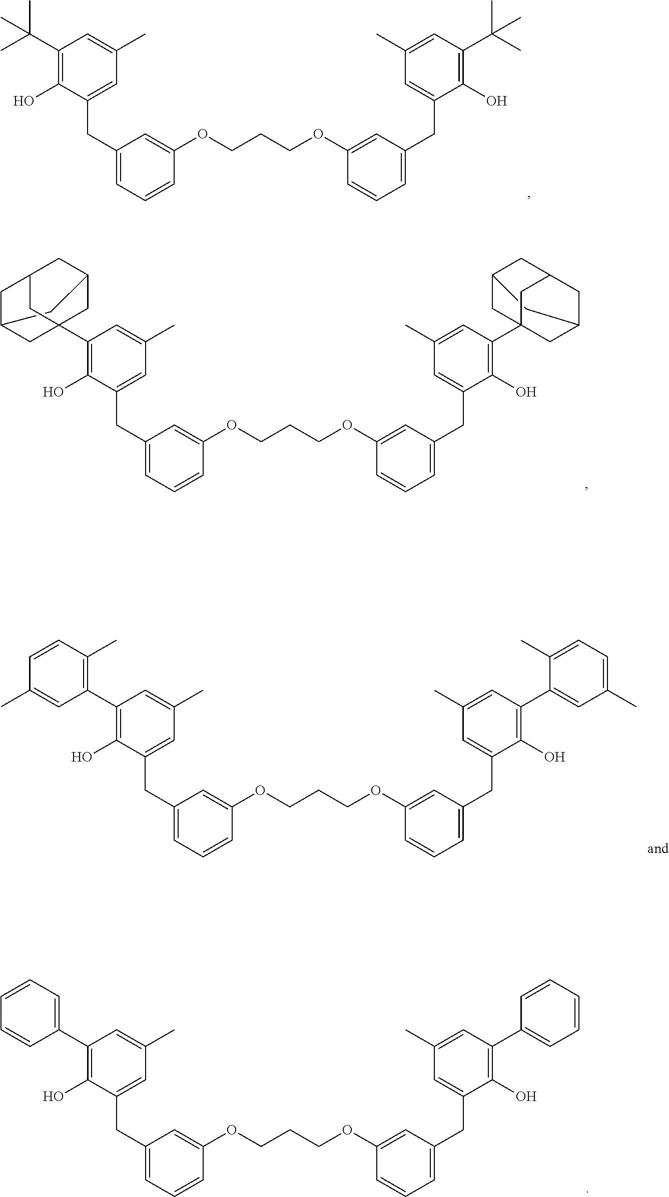

15. The method of claim 13, wherein the ring-opening polymerization is conducted at 23° C.

16. The method of claim 13, wherein the PHB has a Tm between 85° C. and 110° C.

17. A compound of structural Formula I 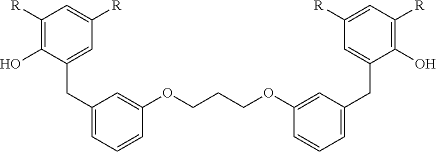
wherein R is selected from the group of hydrogen, alkyl and substituted alkyl.

18. An isolated yttrium complex comprising yttrium and a ligand comprising a compound of the structural Formula I 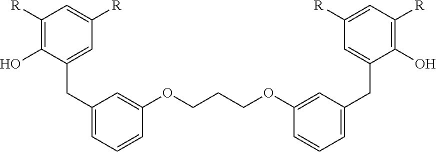
wherein R is selected from the group of hydrogen, alkyl and substituted alkyl.

19. A catalyst system comprising the isolated yttrium complex of claim 18.

20. The catalyst system of claim 19 wherein polymerization is initiated through an alcohol and amine exchange.
